(19) United States
(12) Patent Application Publication
(10) Pub. No.: US 2013/0281544 A1
(43) Pub. Date: Oct. 24, 2013

(54) HEPATOPROTECTANT ACTIVITY OF GARCINOL

(71) Applicants: Muhammed Majeed, Edison, NJ (US); Sarang Bani, Bangalore (IN)

(72) Inventors: Muhammed Majeed, Edison, NJ (US); Sarang Bani, Bangalore (IN)

(21) Appl. No.: 13/885,781

(22) PCT Filed: Dec. 29, 2011
(86) PCT No.: PCT/US11/67733

§ 371 Date: May 16, 2013

Related U.S. Application Data
(60) Provisional application No. 61/428,643, filed on Dec. 30, 2010.

(51) Int. Cl. A61K 31/122

(52) U.S. Cl. CPC A61K 31/122
USPC 514/681; 435/375

(57) ABSTRACT

The present invention discloses the hepatoprotective potential of garcinol.

This application is a non-provisional application of provisional application 61/428,643 filed on Dec. 30, 2010.

### FIELD OF THE INVENTION

The invention in general relates to medicaments for hepatotoxicity (hepatic toxicity) management. More specifically, it relates to the hepatoprotective potential of garcinol.

### DESCRIPTION OF PRIOR ART

The term “hepatotoxicity” refers in general to the chemical induced liver damage. Such damage occurs mainly when the liver discharges its innate function of transformation and clearance of chemicals in the body.

The close association of the liver to the gastrointestinal tract and the spleen through the portal venous system, as part of its metabolic function further exacerbates the impact of toxicity due to circulating drugs.

Nilesh Mehta et al (Drug-Induced Hepatotoxicity—Medscape REFERENCE—Drugs, Diseases and Procedures) reports that 75% of the idiosyncratic drug reactions lead to liver transplantation or death. The same authors also report that in the United States, approximately 2000 cases of acute liver failure occur annually and 50% of such cases could be attributed to the effects of drugs. The authors further elucidate the pathophysiological mechanisms underlying drug induced hepatotoxicity. These include,
(i) Hepatocyte disruption that follows actin fibril disassembly caused due to the covalent binding of the drug to the intracellular proteins and the decrease in ATP levels.
(ii) Drug induced blockage of transport pumps preventing normal bile excretion to cause cholestasis.
(iii) Immune response mediated by the binding of the drug to P-450 enzyme.
(iv) TNF-α mediated apoptosis of hepatocytes.
(v) Mitochondrial disruption due to decreased ATP production brought about by the drug induced inhibition of NAD and FAD in the beta-oxidation energy production mechanism.
(vi) Toxic metabolites induced bile duct injury.
(vii) Activation of liver cell types like the Browicz-Kuppfer cells and receptors like toll-like receptor 4 (TLR4) and CD14 thereof during early ethanol induced liver damage, leading to internalization of the lipopolysaccaride fraction of the cell walls of gram negative bacteria that flourish in the gut in an alcohol rich environment. This then leads to activation of pro-inflammatory cytokines like TNF-α and superoxides which would enter the stellate cells in the liver, leading to collagen synthesis, fibrosis and eventually liver cirrhosis.

Hepatoprotection as an ongoing therapeutic (both preventive and prophylactic) means, thus assumes tremendous importance in conditions where there is interplay of one or more of the aforesaid mechanisms in causing liver damage. Such conditions include,
(i) Nonalcoholic steatohepatitis (NASH) which represents fat in the liver and ensuing inflammation thereof. Though it resembles alcoholic liver disease, it occurs in people who are non-alcoholics or who consume very little alcohol. NASH is enigmatic in the sense that it may regress on its own or worsen in a slow manner leading to fibrosis and subsequently life threatening cirrhosis. Further, no specific therapies exist for this condition except life style management methods.
(ii) Alcohol abuse resulting from habitual or prolonged consumption;
(iii) Chemotherapy for cancer.
(iv) Conditions such as non-alcoholic fatty liver disease where there is fat deposition in the liver without signs and symptoms of inflammation or liver damage.
(v) Viral induced acute or chronic hepatitis, where the latter condition may lead to fibrosis and life threatening cirrhosis/liver cancer.

Garcinol, isolated from Garcinia sp. fruit rind is known in the art as an anti-oxidant and chemo protective agent. (Tanaka, T. et. al. Prevention of colonic aberrant crypt foci by dietary feeding of garcinol in male F3444 rats. Carcinogenesis, June 2000: 21 (6): 1183-9).

Garcinol and isogarcinol were evaluated for their antibacterial activity against methicillin-resistant Staphylococcus aureus (Linuma M et al, Antibacterial activity of some Garcinia benzophenone derivatives against methicillin-resistant Staphylococcus aureus. Biol Pharm Bull 1996 February; 19(2): 311-4).

Garcinol's role as a potent inhibitor of histone acetyltransferases (HATs) both in vitro and in vivo was reported by Tapas et al in 2004 (“Polyisoprenylated Benzophenone, Garcinol, a Natural Histone Acetyl transferase Inhibitor, Represses Chromatin Transcription and Alters Global GeneExpression”, The Journal of Biological Chemistry, Vol. 279, No. 32, Issue of August 6, pp. 33716-33726, 2004).

The present inventors add further to the medical potential of garcinol in disclosing the molecule's hepatoprotectant activity through modulation of one or more pathophysiological effects highlighted herein above. In specific, the present inventors have sought to study the effects of garcinol in modulating the biochemical markers associated with hepatotoxicity.

### SUMMARY OF THE INVENTION

The present invention discloses the potential of garcinol as a hepatoprotectant. Protection of cultures of Hep-2 cells in the presence of effective concentrations of garcinol has been demonstrated. Also demonstrated is garcinol mediated modulation of biochemical markers in animal models of toxin-CCl₄ induced, drug-Paracetamol induced and alcohol induced hepatoxicity.

### BRIEF DESCRIPTION OF DRAWINGS

FIG. 1: shows the graphical representation of the effect of different doses of garcinol on TGF-beta 1 expression (pg/ml) in the liver homogenates from toxin (CCl₄) induced hepatotoxic animal models. Values are expressed as Mean±SE, n=6); P value *: <0.001.

FIG. 2: shows the graphical representation of the effect of different doses of garcinol on Inter-Cellular Adhesion Molecule 1 (ICAM-1 or CD52) expression (pg/ml) in the liver homogenates from toxin (CCl₄) induced hepatotoxic animal models. Values are expressed as Mean±SE, n=6); P value *: <0.001.

FIG. 3: shows the graphical representation of the effect of different doses of garcinol on C-reactive protein expression (mg/ml) in the liver homogenates from toxin (CC₄) induced hepatotoxic animal models. Values are expressed as Mean±SE, n=6); P value *: <0.001.

FIG. 4: shows the graphical representation (flow cytometric studies) of the effect of different doses of garcinol on Tumor Necrosis Factor-α (TNF-α) in the blood drawn from toxin (CCl₄) induced hepatotoxic animal models.

FIG. 5: shows the graphical representation (flow cytometric studies) of the effect of different doses of garcinol on Interleukin-2 (IL-2) in the blood drawn from toxin (CCl₄) induced hepatotoxic animal models.

FIG. 6: shows the graphical representation of the effect of different doses of garcinol on Interleukin-4 (IL-4) in the blood drawn from toxin (CCl₄) induced hepatotoxic animal models.

FIG. 7: shows the graphical representation of the effect of different doses of garcinol on TGF-beta 1 expression (pg/ml) in the liver homogenates from drug (Paracetamol) induced hepatotoxic animal models. Values are expressed as Mean±SE, n=6); P value *: <0.01, **: <0.001. GC: Garcinol; APAP: Paracetamol.

FIG. 8: shows the graphical representation of the effect of different doses of garcinol on Inter-Cellular Adhesion Molecule 1 (ICAM-1 or CD52) expression (pg/ml) in the liver homogenates from drug (Paracetamol) induced hepatotoxic animal models. Values are expressed as Mean±SE, n=6); P value *: <0.01, **: <0.001. GC: Garcinol; APAP: Paracetamol.

FIG. 9: shows the graphical representation (flow cytometric studies) of the effect of different doses of garcinol on Tumor Necrosis Factor-α (TNF-α) in the blood drawn from drug (Paracetamol) induced hepatotoxic animal models.

FIG. 10: shows the graphical representation of the effect of different doses of garcinol on Tumor Necrosis Factor-α (TNF-α) and Interleukin-1β (IL-1β) in the serum of alcohol (Ethyl alcohol) induced hepatotoxic animal models. Values are expressed as Mean±SE, n=6); P value *: <0.01, **: <0.001, GC: Garcinol.

FIG. 11: shows the graphical representation of the effect of different doses of garcinol on Interleukin-12 (IL-12) in the serum of alcohol (Ethyl alcohol) induced hepatotoxic animal models.

FIG. 12: shows the graphical representation of the cytotoxic potential (% cytotoxicity) of garcinol in Hep G2 liver cancer cell line in comparison with Silymarin, a known hepatoprotective agent.

FIG. 13: shows the graphical representation of the hepatoprotective effect of garcinol in comparison with the known hepatoprotective agent Silymarin in Hep G2 liver cancer cell line.

### DETAILED DESCRIPTION

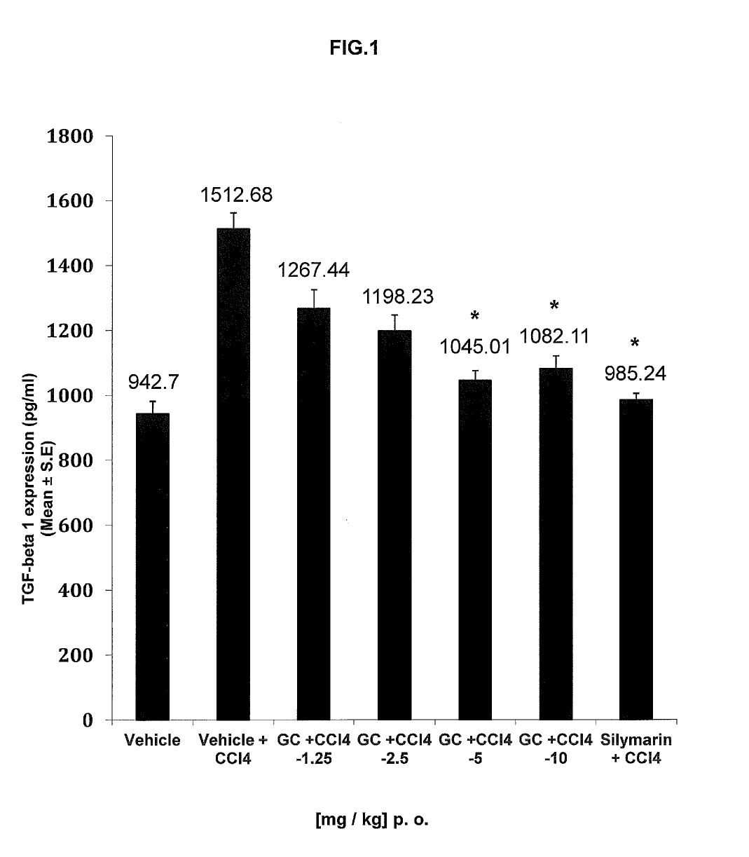

### FIGS. 1-13

### Preferred Embodiment

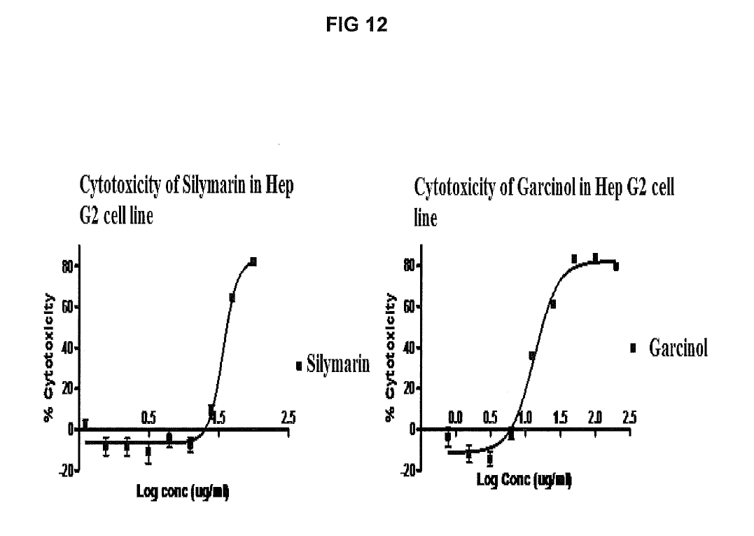

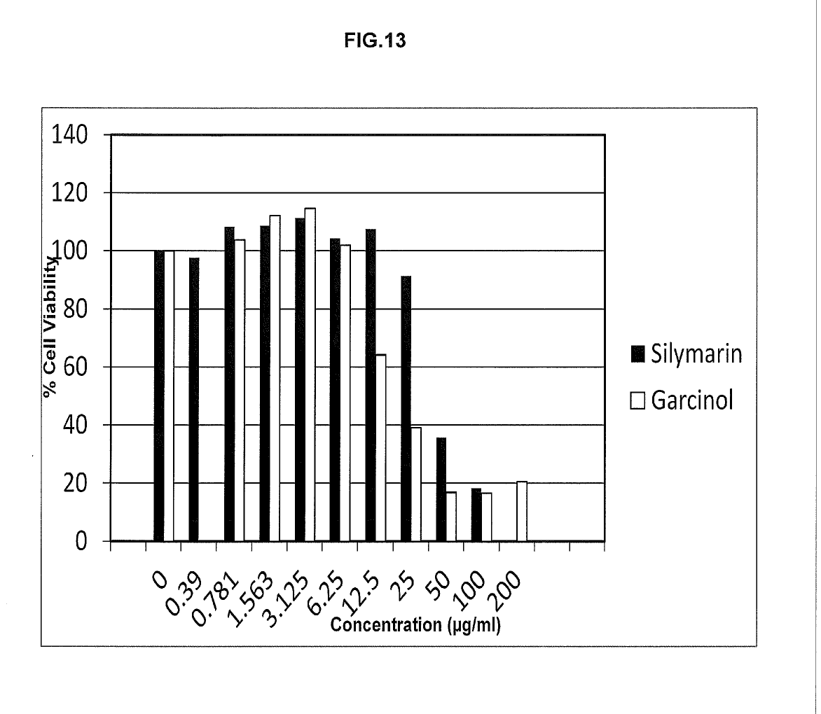

In the most preferred embodiment, the present invention pertains to a method of mammalian hepatocyte protection, said method comprising step of bringing into contact mammalian hepatocytes with an effective concentration of garcinol. More specifically, the effective concentration of garcinol is from about 0.78 μg/ml to about 6.25 μg/ml (FIGS. 12 and 13). In an alternate most preferred embodiment, the present invention relates to a method of providing hepatoprotection, said method comprising step of administering a therapeutically effective amount of garcinol to a subject in need thereof. More specifically, the subject is a mammal.

In another preferred embodiment, the present invention relates to a method of reducing increased levels of cytokine expression in mammalian models of liver damage (hepatotoxicity), said method comprising step of administering an effective amount of garcinol to said models (FIGS. 1, 4, 5, 6, 7, 9, 10 and 11). In specific embodiments, the cytokines are Transforming Growth Factor G-β1 (TGF-β1), Tumor Necrosis Factor-α, Interleukin-2 (IL-2), Interleukin-4 (IL-4) and Interleukin-12 (IL-12). In further specific embodiment, hepatotoxicity in mammalian models may be induced by toxin, drug or ethyl alcohol. In an alternate specific embodiment, liver damage is induced by combinations of toxin, drug and ethyl alcohol.

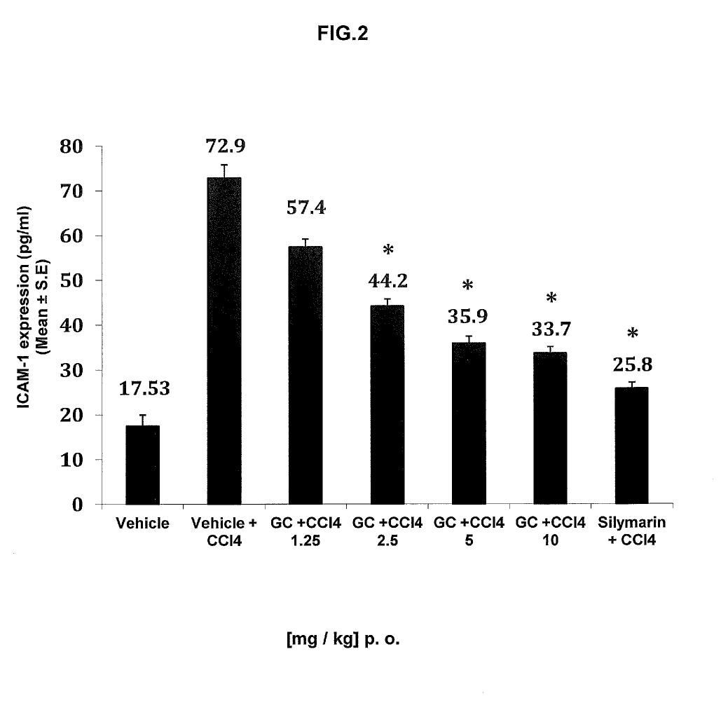

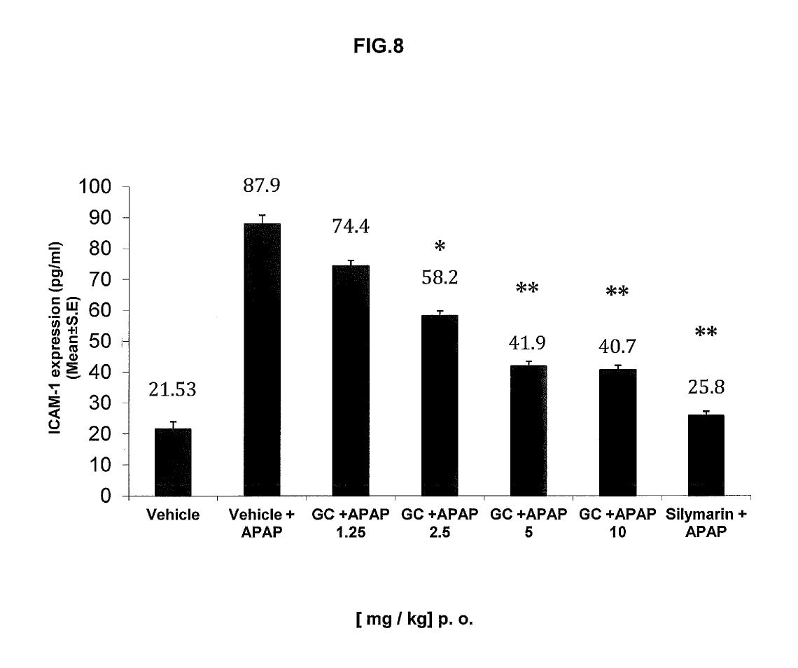

In yet another preferred embodiment, the present invention relates to a method of reducing increased levels of adhesion molecule expression in mammalian models of liver damage (hepatotoxicity) induced by toxins and/or drugs, said method comprising step of administering an effective amount of garcinol to said models (FIGS. 2 and 8). In a specific embodiment, the adhesion molecule is Intracellular Adhesion Molecule-1 (ICAM-1 or CD 52).

In yet another preferred embodiment, the present invention relates to a method of reducing elevated levels of liver enzymes and/or bile pigments in mammalian models of liver damage (hepatotoxicity), said method comprising step of administering an effective amount of garcinol to said models. In specific embodiments, the liver enzymes are Alanine Transaminase, Aspartate aminotransferase and Alkaline phosphatase (Tables 3, 4 and 5). In another specific embodiment, the bile pigment is bilirubin. In further specific embodiment, liver damage in mammalian models may be induced by toxin, drug or ethyl alcohol. In an alternate specific embodiment, liver damage is induced by combinations of toxin, drug and ethyl alcohol.

The potential therapeutic value of garcinol as a hepatoprotectant may be understood through examples elucidated herein below.

### Example 1

Acute Oral Safety of Garcinol: No mortality was observed up to 2000 mg/kg. p. o. in mice up to two weeks of observation. The parameters studied and observations recorded are included in Table 1. (OECD Guidelines for Testing of Chemicals. Guideline 423, Acute Oral Toxicity—Acute Toxic Class Method, Adopted, (1996). Organization for Economic Cooperation and Development).

TABLE 1
Parameters studied on acute oral safety of garcinol

| General Behavior | Dermal |
|---|---|
| Aggressiveness: Nil | Blanching: Nil |
| Fearful: Nil | Hyperemia: Nil |
| Passive: Nil | Cyanosis: Nil |
| General Movement: Normal | |
| General Locomotor Activity: Normal | |
| Central Nervous System | General Observations |
| Excitation: Nil | Muscular Weakness: Nil |
| Motor Activity: Normal | Salivation: Normal |
| Tremors: Nil | Pilo erection: Nil |
| Clonic convulsions: Nil | Diarrhea: Nil |
| Respiratory System | Reflexes |
| Respiration rate: Normal | Corneal: No Effect |
| Respiration Depth: Normal | Pinnal: No Effect |
| Autonomic Nervous System | Food and Water (Intake/Excretion) |
| Motor Activity: Normal | Fecal Output: Normal |
| Atexia: Nil | Urine Output: Normal |
| Respiration Rate: Normal | |
| Diarrhea: Nil | |

### Example 2

### Effect of Different Doses of Garcinol (GC) on TGF-β1, ICAM-1 and CRP Expressions in the Liver Homogenates of Carbon Tetrachloride Induced Liver Damage in Rats

Animals used in the experiment: Male Wistar Rats

Weight of the animals: 140-160 grams

Doses of Garcinol (GC) used for the study: 1.25, 2.5, 5, 10 mg/kg p.o.

Standard drug: Silymarin (50 mg/kg) p.o.

Procedure:

Liver injury was induced by administration of carbon tetrachloride (CCl₄) mixed with liquid paraffin (5 fold dilution). Animals were given single dose of CCl₄ at 1 ml/kg, p.o. followed by the administration of garcinol at different time intervals (Table 2). [(i) Bramanti G, Murmann W, Pierini P and Comporti M (1978). Effect of cicloxilic acid on CCl₄-induced liver injury. Drug Research 28: 1112-1217. (ii) B Singh, A K Saxena, B K Chandan, K K Anand (1998). Hepatoprotective activity of Verbenalin on experimental liver damage in rodents. Fitoterapia LXIX 2: 135-140. (iii) B. K. Chandan, A. K. Sharma, K. K. Anand Boerhaavia diffusa: A study of its hepatoprotective activity. Journal of Ethnopharmacology Volume 31, Issue 3, March 1991, Pages 299-307].

TABLE 2
provides details of day and time schedule of
CCl₄ and garcinol administration

| Day and time | | | |
|---|---|---|---|
| Schedule of | | | |
| toxin/drug (GC) | | | Time elapsed |
| Administration | | Treatment | since toxin administration |
| Day 1 | 10 A.M. | CCl₄ | 0 h |
| | 4 P.M. | GC | 6 h after CCl₄ |
| Day 2 | 10 A.M. | GC | 24 h after CCl₄ |
| Day 3 | 10 A.M. | GC | 48 h after CCl₄ |
| | 12 Noon | Samples Collection | 50 h after CCl₄/2 h after last |
| | | (liver & blood) | treatment of test material |
| | | | (GC) |

Liver tissue was homogenized on ice with a polytron and homogenate was centrifuged at 5000 g for 15 min. Aliquots of the supernatant were separated and used for biochemical analysis. Supernatants were stored at −80° C. until cytokine analysis. TGFβ, ICAM-1, C-reactive protein (CRP) were estimated using commercially available kits based on sandwich and competitive ELISA technique according to the manufacturers' instructions. All cytokine concentrations were carried out by means of colorimetric measurement at 450 nm on an ELISA plate reader by interpolation from a standard curve. [(i) Magari K, Miyata S, Ohkubo Y, Mutoh S, (2004). Inflammatory cytokine levels in paw tissues during development of rat collagen-induced arthritis: Effect of FK506, an inhibitor of T cell activation. Inflammation Research. 53: 469-474 (Magari et al., 2004); and (ii) Modulation of Th1/Th2 cytokines and inflammatory mediators by hydroxychavicol in adjuvant induced arthritic tissues. Anjali Pandey , Sarang Bani, Prabhu Dutt, Krishna Avtar Suri. Cytokine 49 (2010) 114-121]

Results:

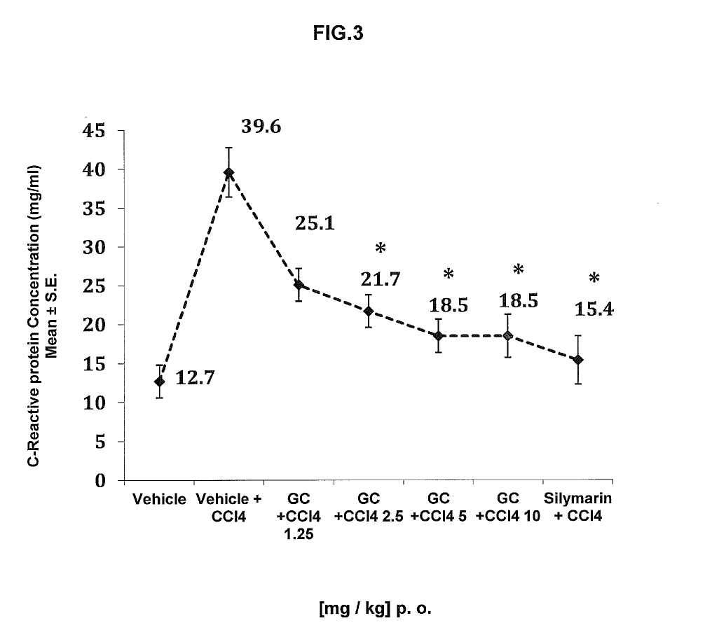

Garcinol inhibited increased levels of TGFβ (FIG. 1) ICAM-1 (FIG. 2), C-reactive protein (CRP) (FIG. 3) associated with acute hepatitis resulting from carbon tetrachloride induced liver damage in rats.

### Example 3

### Effect of Different Doses of Garcinol (GC) on TNF-α, IL-2 and IL-4 in the Blood of Carbon Tetrachloride Induced Liver Damage in Rats

Animals used in the experiment: Male Wistar Rats

Weight of the animals: 140-160 grams

Doses of Garcinol (GC) used for the study: 1.25, 2.5, 5, 10 mg/kg p.o.

Standard drug: Silymarin (50 mg/kg) p.o.

Procedure:

Liver injury was induced by administration of carbon tetrachloride (CCl₄) mixed with liquid paraffin (5 fold dilution). Animals were given single dose of CCl₄ at 1 ml/kg, p.o. followed by the administration of garcinol (See aforesaid Table 2). [(i) Bramanti G, Murmann W, Pierini P and Comporti M (1978). Effect of cicloxilic acid on CCl₄-induced liver injury. Drug Research 28: 1112-1217. (ii) B Singh, A K Saxena, B K Chandan, K K Anand (1998). Hepatoprotective activity of Verbenalin on experimental liver damage in rodents. Fitoterapia LXIX 2: 135-140. (iii) B. K. Chandan, A. K. Sharma, K. K. Anand Boerhaavia diffsa: A study of its hepatoprotective activity. Journal of Ethnopharmacology, Volume 31, Issue 3, March 1991, Pages 299-307].

The animals were bled retro-orbitally and blood was collected in EDTA-coated tubes for the estimation of PE-labeled anti-rat TNF-alpha, IL-2 ad IL-4 monoclonal antibody expression. Analysis was done on flow cytometer (BD-FACS CANTO II). These fluorochrome-labeled monoclonal antibodies were added directly to 100 μl of whole blood, which was then lysed using whole blood lysing reagent. Following the final centrifugation, samples were resuspended in phosphate-buffered saline (pH, 7.4) and analyzed directly on the flow cytometer. A fluorescence trigger was set on the PE (FL2) parameter to collect the events. [(i) Bani S, Kaul A, Khan B, Ahmad S F, Suri K A, Gupta B D, Satti N K, Qazi G N, (2006). Suppression of T lymphocyte activity by lupeol isolated from Crataeva religiosa. Phytotherapy Research; 20(4): 279-287. And (ii) Bani S, Kaul A, Khan B, Ahmad S F, Suri K A, Satti N K, (2005). Immunosuppressive properties of an ethyl acetate fraction from Euphorbia royleana. Journal of Ethnopharmacology; 99: 185-192].

Results:

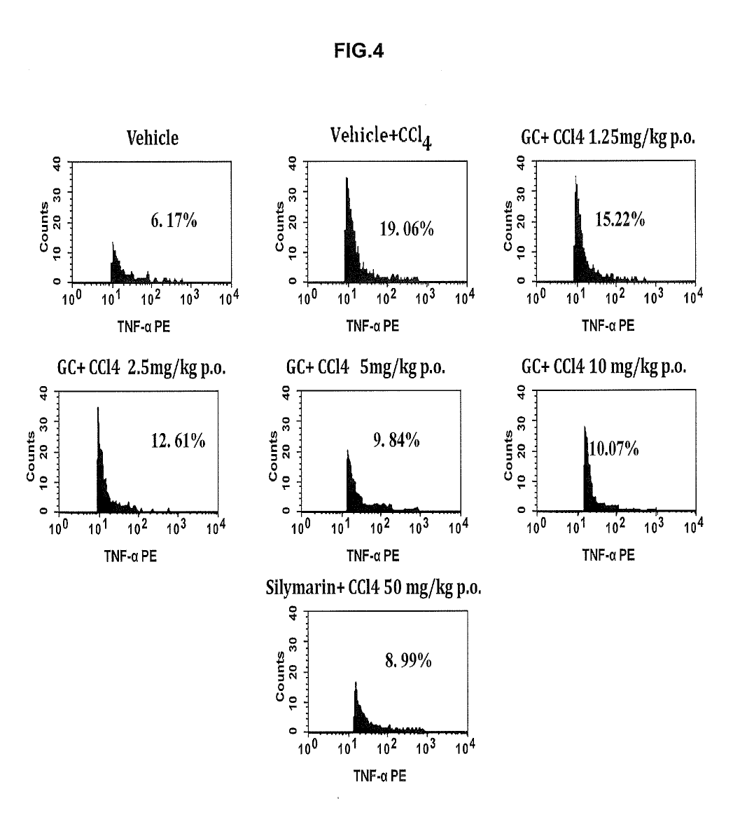

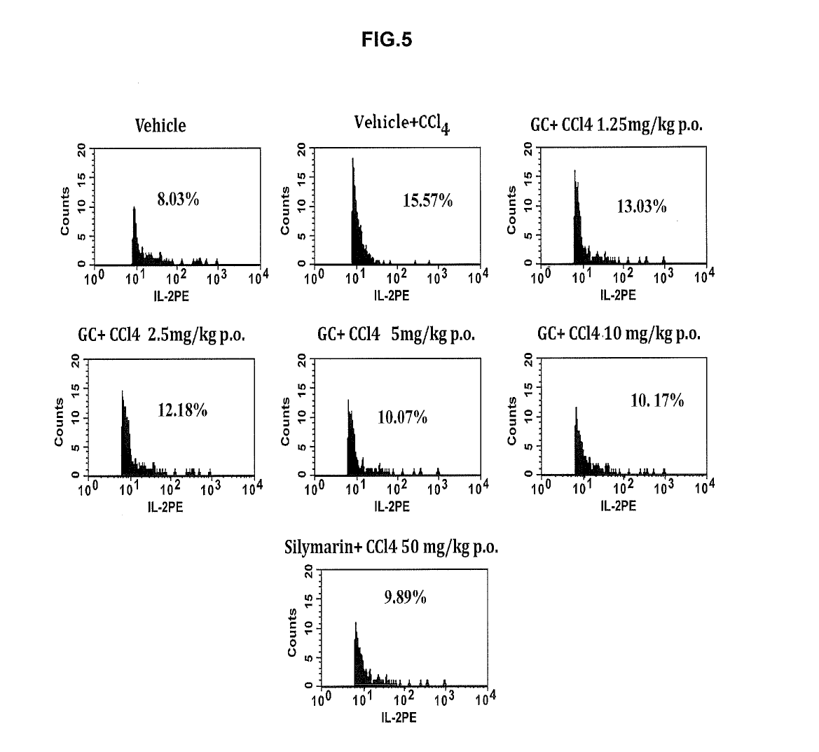

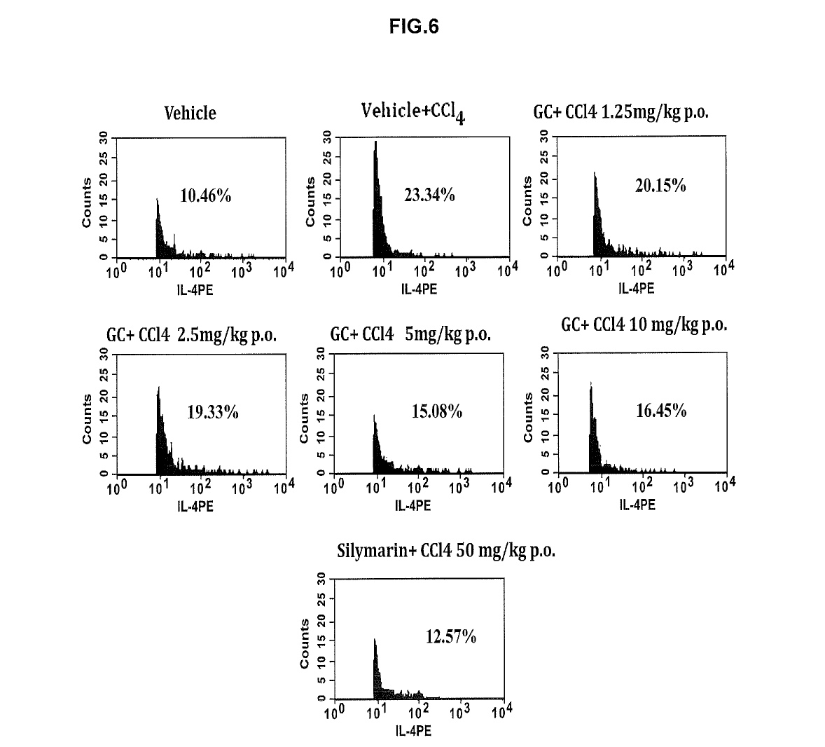

Garcinol (GC) caused a dose based inhibition of T-Cell immune response marked by inhibition of TNF-α (FIG. 4), IL-2 (FIG. 5) and IL-4 (FIG. 6) expressed in the blood of carbon tetrachloride induced liver damage in rats.

### Example 4

### Effect of Different Doses of Garcinol (GC) on Bilirubin and Serum Enzymes Alanine Transaminase (ALT), Aspartate Aminotransferase (AST) and Alkaline Phosphatase (ALP) in Carbon Tetrachloride Induced Liver Damage in Rats

Animals used in the experiment: Male Wistar Rats

Weight of the animals: 140-160 grams

Doses of Garcinol (GC) used for the study: 1.25, 2.5, 5, 10 mg/kg p.o.

Standard drug: Silymarin (50 mg/kg) p.o.

Procedure:

Liver injury was induced by administration of carbon tetrachloride (CCl₄) mixed with liquid paraffin (5 fold dilution). Animals were given single dose of CCl₄ at 1 ml/kg, p.o. followed by the administration of garcinol (Table 2). [(i) Bramanti G, Murmann W, Pierini P and Comporti M (1978). Effect of cicloxilic acid on CCl₄-induced liver injury. Drug Research 28: 1112-1217. (ii) B Singh, A K Saxena, B K Chandan, K K Anand (1998). Hepatoprotective activity of Verbenalin on experimental liver damage in rodents. Fitoterapia LXIX 2: 135-140. (iii) B. K. Chandan, A. K. Sharma, K. K. Anand Boerhaavia difiusa: A study of its hepatoprotective activity. Journal of Ethnopharmacology, Volume 31, Issue 3, March 1991, Pages 299-307].

Blood was collected from the retro-orbital plexus of experimental animals and no anti-coagulant was added. Blood was made to stand at room temperature for 1 hour. It was then centrifuged and clear serum was separated. The serum was then stored for analysis. Reference: (Magari et al., 2004).

Serum Biochemistry I

(ASPARTATE AMINOTRANSFERASE and ALANINE AMINOTRANSFERASE), Reference: Ritman S, Frankel S, (1957). A colorimeteric method for the determination of serum glutamic oxaloacetic and glutamic pyruvic transaminases. American Journal of Clinical Pathology; 28: 56-63.

Procedure:

0.2 ml of serum, collected from test and control samples, was mixed with 1.0 ml of buffer solution and incubated for 60 min at 37° C. in a water bath. After the addition of 1.0 ml of chromogen solution, the samples were kept at room temperature for 20 minutes for the reaction to proceed and 10 ml NaOH was added. The optical density was red at 546 nm after 5 min. To the blank, serum was added after the addition of chromogen solution.

Serum Biochemistry II

(ALKALINE PHOSPHATASE), Reference: Klaus Walter and Schutt C. (1974). Acid and alkaline phosphatase in serum. In: Methods of Enzymatic Analysis. Eds. Hans Ulrich Bergmeyer, Verlag ChemieWeinheim, Academic press, Inc. New York, 2^{nd} Edition, Vol. 2, pp. 855-64.

Procedure:

Alkaline phosphatase activity measurement is based on the ability of the enzyme to hydrolyze p-nitrophenol phosphate under alkaline conditions. The cleaved product p-nitrophenol is yellow in alkaline solution and is measured at 400-420 nm (Klaus and Schutt, 1974). 2.0 ml of buffered substrate was taken in the tubes ‘Test’ ‘Control’ and ‘Blank’ followed by addition of serum and distilled water (0.1 ml) in ‘Test’ and ‘Blank’ respectively and incubated in a water bath for 30 minutes at 25° C. After incubation sodium hydroxide was added (0.25 N, 2 ml) to all the tubes, which was followed by serum (0.1 ml) to the tubes marked ‘Control’. The yellow colour formed was measured spectrophotometrically against blank at 410 nm.

Serum Biochemistry III

[BILIRUBIN (BRBN)], Reference: Malloy H T and Evelyn K A (1937). The determination of bilirubin with photoelectric colorimeter. Journal of Biological Chemistry 119: 481-490.

Procedure:

Serum bilirubin was estimated by the method of Malloy and Evelyn (1937). In the two sets of test tubes marked ‘Test’ and ‘Control’, serum (0.2 ml) and distilled water (1.8 ml) were added. To the tubes marked ‘Test’ and ‘Standard’ diazo reagent (0.5 ml) was added. To the test tubes marked ‘Control’ and ‘Blank’ diazo blank (0.5 ml) was added. Finally methanol (2.5 ml) was added into each test tube. The tubes were mixed and allowed to stand for 30 minutes in dark. The tubes were read after 10 minutes at 540 nm.

Results:

Garcinol reduced increased levels of bilirubin and serum enzymes Alanine Transaminase (ALT), Aspartate Aminotransferase (AST) and Alkaline Phosphatase (ALP) in carbon tetrachloride induced liver damage in rats (Table 3).

TABLE 3
Effect of Garcinol on bilirubin, ALT, AST and ALP in
carbon tetrachloride induced liver damage in rats
SERUM PARAMETERS

| | | | | | | Average |
|---|---|---|---|---|---|---|
| | Dose | ALT | AST | ALP | Bilirubin | Protection |
| Treatments | (mg/kg) | μmol/min/lt | μmol/min/lt | μmol/min/lt | mg % | % |
| Vehicle | — | 110.30 ± 13.70 | 107.70 ± 14.00 | 30.90 ± 0.72 | 0.40 ± 0.05 | — |
| Vehicle + | — | 1460.03 ± 78.30 | 878.10 ± 41.52 | 87.99 ± 3.35 | 1.17 ± 0.07 | — |
| CCl₄ | | | | | | |
| GC + CCl₄ | 1.25 | 1272.50 ± 109.19 | 746.81 ± 36.93 | 78.49 ± 6.58 | 1.03 ± 0.04 | 12.63 |
| | | (12.84) | (14.95) | (10.79) | (11.96) | |
| GC + CCl₄ | 2.50 | 1027.07 ± 68.26* | 646.90 ± 50.53* | 70.32 ± 3.89* | 0.89 ± 0.05** | 24.99 |
| | | (29.65) | (26.32) | (20.08) | (23.93) | |
| GC + CCl4 | 5.00 | 828.59 ± 46.56** | 500.66 ± 49.22** | 59.19 ± 4.45** | 0.78 ± 0.04** | 38.07 |
| | | (43.24) | (42.98) | (32.73) | (33.33) | |
| GC + CCl4 | 10.00 | 891.36 ± 45.72** | 522.43 ± 57.50** | 59.70 ± 3.69** | 0.75 ± 0.04** | 36.87 |
| | | (38.94) | (40.50) | (32.15) | (35.89) | |
| Silymarin + | 50 | 744.76 ± 40.16** | 460.76 ± 25.60** | 49.84 ± 2.80** | 0.64 ± 0.04** | 47.14 |
| CCl₄ | | (48.99) | (47.52) | (43.35) | (48.71) | |

(Values as Mean ± SE, n = 6); Percent change in parenthesis; P value *<0.01; **<0.001. (ALT) Alanine transaminase; (AST) Aspartate aminotransferase; (ALP) Alkaline phosphatase.

### Example 5

### Effect of Different Doses of Garcinol (GC) on TGF-beta 1 and ICAM-1 Expression in Liver Homogenate of Paracetamol Induced Liver Damage in Rats

Animals used in the experiment: Male Wistar Rats

Weight of the animals: 140-160 grams

Doses of Garcinol (GC) used for the study: 1.25, 2.5, 5, 10 mg/kg p.o.

Standard drug: Silymarin (50 mg/kg) p. o.

Procedure:

Experimental animals orally received paracetamol (400 mg/kg body weight) for seven days. The animals from drug treated group received 400 mg/kg body weight of paracetamol dissolved in water orally along with graded doses of test drug Garcinol for seven days. The standard group animals received 50 mg/kg body weight of standard drug silymarin and 400 mg/kg body weight of paracetamol for seven days and served as standard control. [N. Kanchana and A. Mohamed Sadiq, Hepatoprotective effect of Plumbago zeylanica on paracetamol induced liver toxicity in rats, Int J Pharm Pharm Sci, Vol 3, Issue 1, 151-154 (2011)].

Liver tissue from experimental animals was homogenized on ice with a polytron and homogenate was centrifuged at 5000 g for 15 min. Aliquots of the supernatant were separated and used for biochemical analysis. Supernatants were stored at −80° C. until cytokine analysis. TGFβ and ICAM-1 were estimated using commercially available kits based on sandwich and competitive ELISA technique according to the manufacturers' instructions. All cytokine concentrations were carried out by means of colorimetric measurement at 450 nm on an ELISA plate reader by interpolation from a standard curve. [(i) Magari K, Miyata S, Ohkubo Y, Mutoh S, (2004). Inflammatory cytokine levels in paw tissues during development of rat collagen-induced arthritis: Effect of FK506, an inhibitor of T cell activation. Inflammation Research. 53: 469-474 (Magari et al., 2004); and (ii) Modulation of Th1/Th2 cytokines and inflammatory mediators by hydroxychavicol in adjuvant induced arthritic tissues. Anjali Pandey, Sarang Bani, Prabhu Dutt, Krishna Avtar Suri. Cytokine 49 (2010) 114-121].

Results:

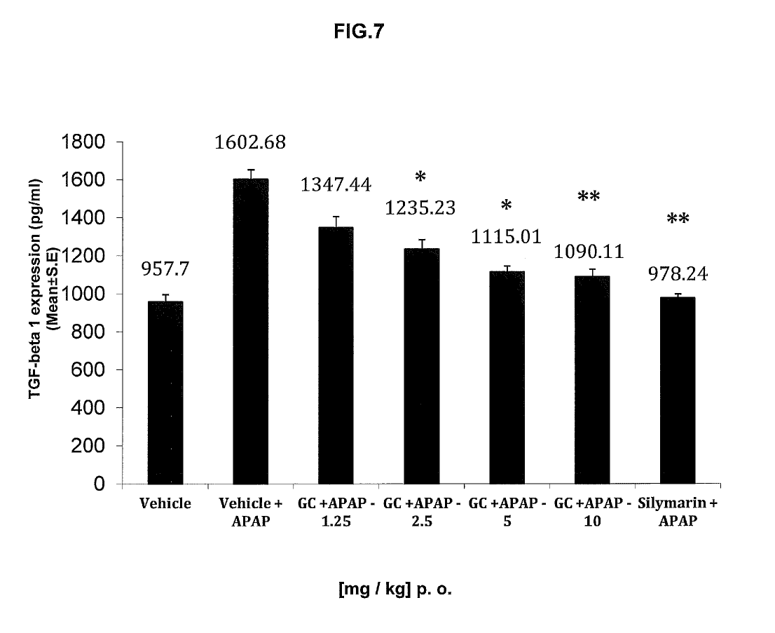

Garcinol caused a dose dependent inhibition of increased levels of TGF-beta and ICAM-1 expressions in the liver associated with paracetamol induced acute hepatitis (FIG. 7 and FIG. 8).

### Example 6

### Effect of Different Doses of Garcinol (GC) on Serum Enzymes Alanine Transaminase (ALT), Aspartate Aminotransferase (AST) and Alkaline Phosphatase (ALP) in Paracetamol (Acetyl-Para-Aminophenol-APAP) Induced Liver Damage in Rats

Animals used in the experiment: Male Wistar Rats

Weight of the animals: 140-160 grams

Doses of Garcinol (GC) used for the study: 1.25, 2.5, 5, 10 mg/kg p.o.

Standard drug: Silymarin (50 mg/kg) p.o.

Procedure:

Experimental animals orally received paracetamol (400 mg/kg body weight) for seven days. The animals from drug treated group received 400 mg/kg body weight of paracetamol dissolved in water orally along with graded doses of test drug Garcinol for seven days. The standard group animals received 50 mg/kg body weight of standard drug silymarin and 400 mg/kg body weight of paracetamol for seven days and served as standard control. [N. Kanchana and A. Mohamed Sadiq, Hepatoprotective effect of Plumbago zeylanica on paracetamol induced liver toxicity in rats, Int J Pharm Pharm Sci, Vol 3, Issue 1, 151-154 (2011)].

Blood was collected from the retro-orbital plexus of experimental animals and no anti-coagulant was added. Blood was made to stand at room temperature for 1 hour. It was then centrifuged and clear serum was separated. The serum was then stored for analysis. Reference: (Magari et al., 2004).

Serum Biochemistry I

(ASPARTATE AMINOTRANSFERASE and ALANINE AMINOTRANSFERASE), Reference: Ritman S, Frankel S, (1957). A colorimeteric method for the determination of serum glutamic oxaloacetic and glutamic pyruvic transaminases. American Journal of Clinical Pathology; 28: 56-63.

Procedure:

0.2 ml of serum, collected from test and control samples, was mixed with 1.0 ml of buffer solution and incubated for 60 min at 37° C. in a water bath. After the addition of 1.0 ml of chromogen solution, the samples were kept at room temperature for 20 minutes for the reaction to proceed and 10 ml NaOH was added. The optical density was red at 546 nm after 5 min. To the blank, serum was added after the addition of chromogen solution.

Serum Biochemistry II

(ALKALINE PHOSPHATASE), Reference: Klaus Walter and Schutt C. (1974). Acid and alkaline phosphatase in serum. In: Methods of Enzymatic Analysis. Eds. Hans Ulrich Bergmeyer, Verlag ChemieWeinheim, Academic press, Inc. New York, 2^{nd} Edition, Vol. 2, pp. 855-64.

Procedure:

Alkaline phosphatase activity measurement is based on the ability of the enzyme to hydrolyze p-nitrophenol phosphate under alkaline conditions. The cleaved product p-nitrophenol is yellow in alkaline solution and is measured at 400-420 nm (Klaus and Schutt, 1974). 2.0 ml of buffered substrate was taken in the tubes ‘Test’ ‘Control’ and ‘Blank’ followed by addition of serum and distilled water (0.1 ml) in ‘Test’ and ‘Blank’ respectively and incubated in a water bath for 30 minutes at 25° C. After incubation sodium hydroxide was added (0.25 N, 2 ml) to all the tubes, which was followed by serum (0.1 ml) to the tubes marked ‘Control’. The yellow colour formed was measured spectrophotometrically against blank at 410 nm.

Serum Biochemistry III

[BILIRUBIN (BRBN)], Reference: Malloy H T and Evelyn K A (1937). The determination of bilirubin with photoelectric colorimeter. Journal of Biological Chemistry 119: 481-490.

Procedure:

Serum bilirubin was estimated by the method of Malloy and Evelyn (1937). In the two sets of test tubes marked ‘Test’ and ‘Control’, serum (0.2 ml) and distilled water (1.8 ml) were added. To the tubes marked ‘Test’ and ‘Standard’ diazo reagent (0.5 ml) was added. To the test tubes marked ‘Control’ and ‘Blank’ diazo blank (0.5 ml) was added. Finally methanol (2.5 ml) was added into each test tube. The tubes were mixed and allowed to stand for 30 minutes in dark. The tubes were read after 10 minutes at 540 nm.

Results:

Garcinol reduced increased levels of bilirubin and serum enzymes Alanine Transaminase (ALT), Aspartate Aminotransferase (AST) and Alkaline Phosphatase (ALP) in paracetamol induced liver damage in rats (Table 4).

TABLE 4
Effect of Garcinol on bilirubin, ALT, AST and ALP in paracetamol induced
liver damage in rats
SERUM PARAMETERS

| | Dose | ALT (μmol | AST (μmol | ALP (μmol | Average |
|---|---|---|---|---|---|
| Treatments | (mg/kg) | min/lt) | min/lt) | min/lt) | Protection % |
| Vehicle | — | 98.32 ± 5.20 | 117.49 ± 8.26 | 21.42 ± 2.04 | — |
| Vehicle + APAP | — | 1102.10 ± 46.98 | 839.79 ± 59.17 | 40.54 ± 3.61 | — |
| GC + APAP | 1.25 | 862.97 ± 53.38 | 761.61 ± 43.96 | 37.21 ± 3.44 | 13.06 |
| | | (21.69) | (9.30) | (8.21) | |
| GC + APAP | 2.50 | 808.64 ± 38.55* | 650.88 ± 35.84* | 34.28 ± 1.98 | 21.51 |
| | | (26.62) | (22.49) | (15.44) | |
| GC + APAP | 5.00 | 661.67 ± 51.40** | 463.41 ± 38.56** | 30.74 ± 2.05** | 36.31 |
| | | (39.96) | (44.81) | (24.17) | |
| GC + APAP | 10.00 | 590.22 ± 50.98** | 536.39 ± 44.13** | 33.60 ± 1.93* | 33.22 |
| | | (46.44) | (36.12) | (17.11) | |
| Silymarin + | 50 | 377.04 ± 14.44** | 379.64 ± 23.22** | 26.90 ± 2.24** | 51.40 |
| APAP | | (65.78) | (54.79) | (33.64) | |

(Values as Mean ± SE, n = 6);
Percent change in parenthesis;
P value
*<0.01;
**<0.001.
(ALT) Alanine transaminase;
(AST) Aspartate aminotransferase;
(ALP) Alkaline phosphatase

### Example 7

### Effect of Different Doses of Garcinol (GC) on TNF-α in the Blood of Paracetamol Induced Liver Damage in Rats

Animals used in the experiment: Male Wistar Rats

Weight of the animals: 140-160 grams

Doses of Garcinol (GC) used for the study: 1.25, 2.5, 5, 10 mg/kg p.o.

Standard drug: Silymarin (50 mg/kg) p.o.

Procedure:

Experimental animals orally received paracetamol (400 mg/kg body weight) for seven days. The animals from drug treated group received 400 mg/kg body weight of paracetamol dissolved in water orally along with graded doses of test drug Garcinol for seven days. The standard group animals received 50 mg/kg body weight of standard drug silymarin and 400 mg/kg body weight of paracetamol for seven days and served as standard control. [N. Kanchana and A. Mohamed Sadiq, Hepatoprotective effect of Plumbago zeylanica on paracetamol induced liver toxicity in rats, Int J Pharm Pharm Sci, Vol 3, Issue 1, 151-154 (2011)].

The animals were bled retro-orbitally and blood was collected in EDTA-coated tubes for the estimation of PE-labeled anti-rat TNF-alpha monoclonal antibody expression. Analysis was done on flow cytometer (BD-FACS CANTO II). These fluorochrome-labeled monoclonal antibodies were added directly to 100 μl of whole blood, which was then lysed using whole blood lysing reagent. Following the final centrifugation, samples were resuspended in phosphate-buffered saline (pH, 7.4) and analyzed directly on the flow cytometer. A fluorescence trigger was set on the PE (FL2) parameter to collect the events. [(i) Bani S, Kaul A, Khan B, Ahmad S F, Suri K A, Gupta B D, Satti N K, Qazi G N, (2006). Suppression of T lymphocyte activity by lupeol isolated from Crataeva religiosa. Phytotherapy Research; 20(4): 279-287. And (ii) Bani S, Kaul A, Khan B, Ahmad S F, Suri K A, Satti N K, (2005). Immunosuppressive properties of an ethyl acetate fraction from Euphorbia royleana. Journal of Ethnopharmacology; 99: 185-192].

Results:

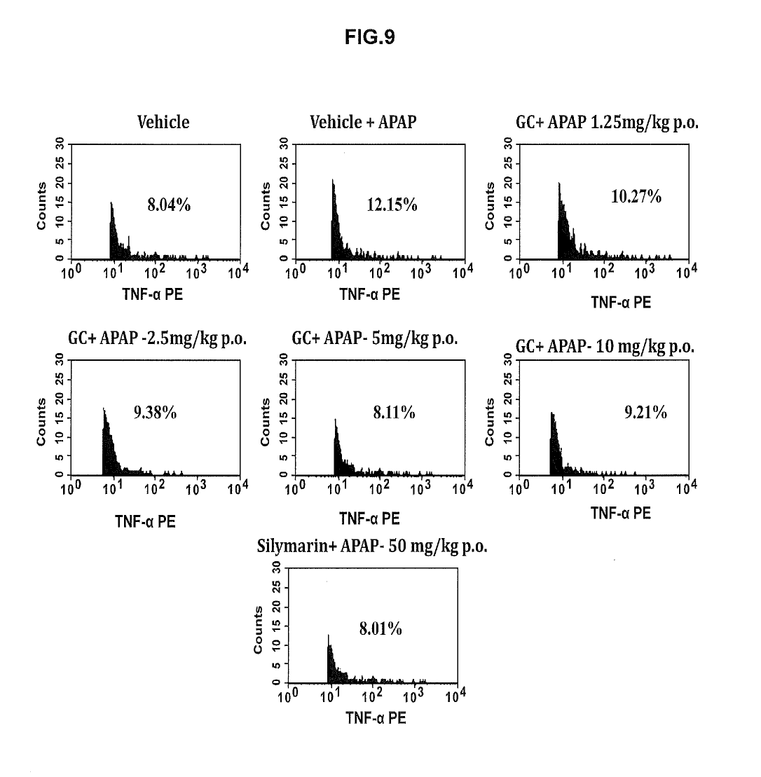

FIG. 9 shows that Garcinol causes dose dependant reduction of increased levels of TNF-α in the liver occurring in acute hepatitis caused by drug (paracetamol) induced liver damage.

### Example 8

### The Effect of Different Doses of Garcinol (GC) on Tumor Necrosis Factor-α (TNF-α), Interleukin-1β (IL-1β) and IL-12 in the Serum of Alcohol (Ethyl Alcohol-EtOH) Induced Hepatotoxic Animal Models

Animals used in the experiment: Male Wistar Rats

Weight of the animals: 140-160 grams

Doses of Garcinol (GC) used for the study: 1.25, 2.5, 5, 10 mg/kg p.o.

Standard drug: Silymarin (50 mg/kg) p.o.

Procedure: Male Wistar rats, weighing 180-200 g, were given 0.5-0.6 ml ethanol orally. The initial dose of ethanol was 6 g/kg/day (solutions maximally containing 56% alcohol), and the dose was progressively increased during week 1 to a maintenance dose of 8 g/kg/day that was continued for 5 more weeks. All rats had regular standard rat chow available throughout the 6-week period. Rats were weighted three times per week. [Guangjin Yuan, Zuojiong Gong *, Xiaorong Zhou, Pin Zhang, Xiaomei Sun and Xi Li. Epigallocatechin-3-Gallate Ameliorates Alcohol-Induced Liver Injury in Rats. Int. J. Mol. Sci. 2006, 7, 204-219].

Blood was collected from the retro-orbital plexus of the alcohol treated experimental animals and mixed with EDTA for cytokine estimations. For collecting serum no anti-coagulant was added to the blood and it was made to stand at room temperature for 1 h. The blood was then centrifuged and clear serum was separated and stored for analysis. TNF-α, IL-1β and IL-12 were estimated using commercially available kits based on sandwich and competitive ELISA technique according to the manufacturers' instructions. All cytokine concentrations were carried out by means of colorimetric measurement at 450 nm on an ELISA plate reader by interpolation from a standard curve.

Result:

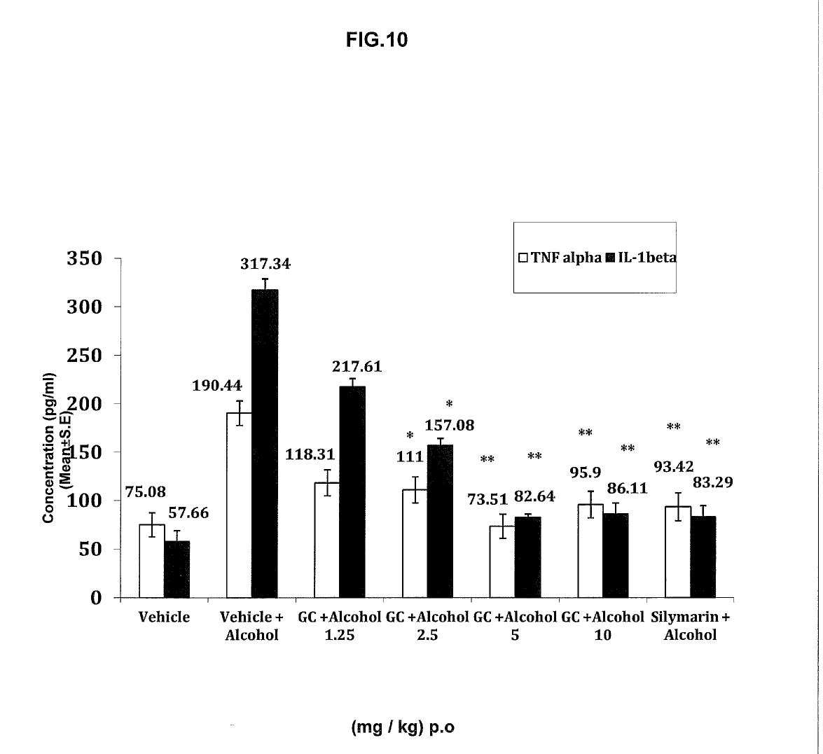

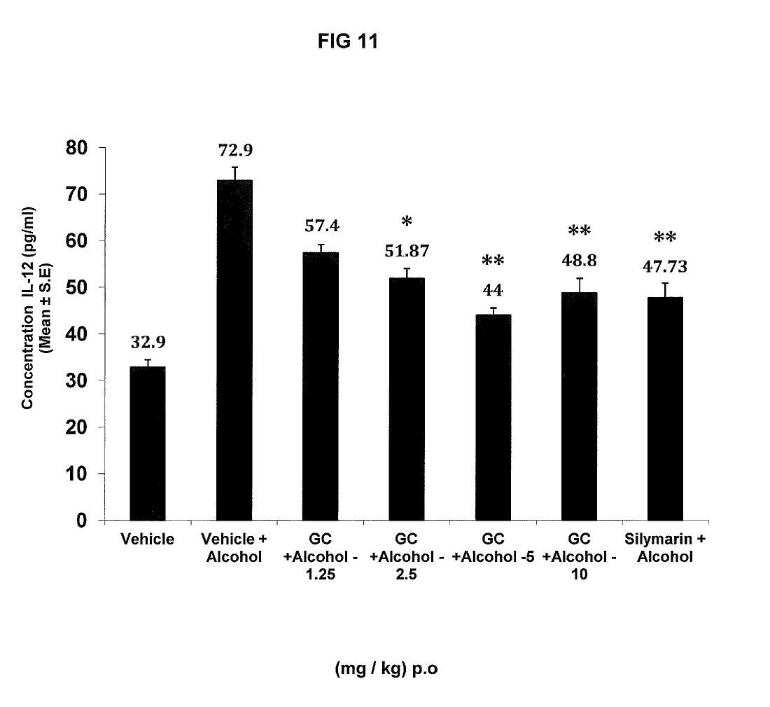

Garcinol (GC) inhibited increased levels of TNF-alpha, Interleukin-1 beta and Interleukin-12 (FIG. 10 and FIG. 11) induced by TLR-4 activation of Kupffer cells by LPS of gram negative bacteria in the gut the activation and excessive growth of which is due to ethyl alcohol intake.

### Example 9

### Effect of Different Doses of Garcinol (GC) on Serum Enzymes Alanine Transaminase (ALT), Aspartate Aminotransferase (AST) and Alkaline Phosphatase (ALP) in the Serum of Alcohol (Ethyl alcohol) Induced Hepatotoxic Animal Models

Animals used in the experiment: Male Wistar Rats

Weight of the animals: 140-160 grams

Doses of Garcinol (GC) used for the study: 1.25, 2.5, 5, 10 mg/kg p.o.

Standard drug: Silymarin (50 mg/kg) p.o.

Procedure:

Male Wistar rats, weighing 180-200 g, were given 0.5-0.6 ml ethanol orally. The initial dose of ethanol was 6 g/kg/day (solutions maximally containing 56% alcohol), and the dose was progressively increased during week 1 to a maintenance dose of 8 g/kg/day that was continued for 5 more weeks. All rats had regular standard rat chow available throughout the 6-week period. Rats were weighted three times per week. [Guangjin Yuan, Zuojiong Gong *, Xiaorong Zhou, Pin Zhang, Xiaomei Sun and Xi Li. Epigallocatechin-3-Gallate Ameliorates Alcohol-Induced Liver Injury in Rats. Int. J. Mol. Sci. 2006, 7, 204-219]. Blood was collected from the retro-orbital plexus of experimental animals and no anti-coagulant was added. Blood was made to stand at room temperature for 1 hour. It was then centrifuged and clear serum was separated. The serum was then stored for analysis. Reference: (Magari et al., 2004).

Serum Biochemistry I

(ASPARTATE AMINOTRANSFERASE and ALANINA AMINOTRANSFERASE), Reference: Ritman S, Frankel S, (1957). A colorimetric method for the determination of serum glutamic oxaloacetic and glutamic pyruvic transaminases. American Journal of Clinical Pathology; 28: 56-63.

Procedure:

0.2 ml of serum, collected from test and control samples, was mixed with 1.0 ml of buffer solution and incubated for 60 min at 37° C. in a water bath. After the addition of 1.0 ml of chromogen solution, the samples were kept at room temperature for 20 minutes for the reaction to proceed and 10 ml NaOH was added. The optical density was red at 546 nm after 5 min. To the blank, serum was added after the addition of chromogen solution.

Serum Biochemistry II

(ALKALINE PHOSPHATASE), Reference: Klaus Walter and Schutt C. (1974). Acid and alkaline phosphatase in serum. In: Methods of Enzymatic Analysis. Eds. Hans Ulrich Bergmeyer, Verlag ChemieWeinheim, Academic press, Inc. New York, 2^{nd} Edition, Vol. 2, pp. 855-64.

Procedure:

Alkaline phosphatase activity measurement is based on the ability of the enzyme to hydrolyze p-nitrophenol phosphate under alkaline conditions. The cleaved product p-nitrophenol is yellow in alkaline solution and is measured at 400-420 nm (Klaus and Schutt, 1974). 2.0 ml of buffered substrate was taken in the tubes ‘Test’ ‘Control’ and ‘Blank’ followed by addition of serum and distilled water (0.1 ml) in ‘Test’ and ‘Blank’ respectively and incubated in a water bath for 30 minutes at 25° C. After incubation sodium hydroxide was added (0.25 N, 2 ml) to all the tubes, which was followed by serum (0.1 ml) to the tubes marked ‘Control’. The yellow colour formed was measured spectrophotometrically against blank at 410 nm.

Serum Biochemistry III

[BILIRUBIN (BRBN)], Reference: Malloy H T and Evelyn K A (1937). The determination of bilirubin with photoelectric colorimeter. Journal of Biological Chemistry 119: 481-490.

Procedure:

Serum bilirubin was estimated by the method of Malloy and Evelyn (1937). In the two sets of test tubes marked ‘Test’ and ‘Control’, serum (0.2 ml) and distilled water (1.8 ml) were added. To the tubes marked ‘Test’ and ‘Standard’ diazo reagent (0.5 ml) was added. To the test tubes marked ‘Control’ and ‘Blank’ diazo blank (0.5 ml) was added. Finally methanol (2.5 ml) was added into each test tube. The tubes were mixed and allowed to stand for 30 minutes in dark. The tubes were read after 10 minutes at 540 nm.

TABLE 5
SERUM PARAMETERS

| | | | | | | Average |
|---|---|---|---|---|---|---|
| | Dose | ALT | AST | ALP | Bilirubin | Protection |
| Treatments | (mg/kg) | (μmol min/lt) | (μmol min/lt) | (μmol min/lt) | mg % | % |
| Vehicle | — | 118.10 ± 10.20 | 106.40 ± 16.60 | 32.30 ± 0.52 | 0.44 ± 0.08 | — |
| Vehicle + | — | 1820.08 ± 42.80 | 1076.32 ± 48.22 | 104.22 ± 8.60 | 2.08 ± 0.02 | — |
| EtOH | | | | | | |
| GC + EtOH | 1.25 | 1320.40 ± 52.20* | 858.80 ± 32.64 | 88.68 ± 8.58 | 1.94 ± 0.02 | 17.32 |
| | | (27.45) | (20.20) | (14.91) | (6.73) | |
| GC + EtOH | 2.50 | 1080.50 ± 48.56** | 726.78 ± 46.50* | 76.12 ± 4.88* | 1.68 ± 0.04* | 29.82 |
| | | (40.63) | (32.47) | (26.96) | (19.23) | |
| GC + EtOH | 5.00 | 876.58 ± 76.80** | 650.26 ± 56.20** | 68.32 ± 5.60** | 1.42 ± 0.06** | 39.39 |
| | | (51.83) | (39.58) | (34.44) | (31.73) | |
| GC + EtOH | 10.00 | 870.80 ± 48.40** | 632.48 ± 44.56** | 69.00 ± 4.82** | 1.39 ± 0.04** | 40.20 |
| | | (52.15) | (41.23) | (33.79) | (33.65) | |
| Silymarin + | 50 | 854.88 ± 38.46** | 662.70 ± 38.40** | 66.54 ± 6.30** | 1.34 ± 0.02** | 40.79 |
| EtOH | | (53.03) | (38.44) | (36.15) | (35.57) | |

(Values as Mean ± SE, n = 6); Percent change in parenthesis; P value *<0.01; **<0.001. (ALT) Alanine transaminase; (AST) Aspartate aminotransferase; (ALP) Alkaline phosphatase

Result:

Garcinol (GC) reduced the increased levels of AST, ALT, ALP and Bilirubin in ethyl alcohol induced hepatotoxic experimental models.

### Example 10

### The Cytotoxic Potential (% Cytotoxicity) of Garcinol in Hep G2 Liver Cancer Cell Line in Comparison with Silymarin, a Known Hepatoprotective Agent (Overall Hepatoprotective Effect of Garcinol vs Silymarin—Tables 6 and 7, FIG. 12 and FIG. 13)

Procedure

HEP G2 cells grown in varying concentrations of the material to be checked for cytotoxicity are taken. The medium is then tapped off gently and 100 ml of working stock solution of MTT (150 mg/well) is added into each well. Then the plate is further wrapped in aluminium foil and incubated for 4 hours in CO₂ incubator at 37° C. The plate is then washed gently with 100 ml of PBS per well. The washing must be done soon after tapping off the medium to avoid drying, flaking and loss of cells during washing. Solubilize the dye in 100 ml of DMSO per well. The plates are shaken for 5 minutes and absorbance read at 492 nm using a Fluostar optima (BMG) micro plate reader. The absorbance will be directly proportional to the cell viability. The option of reading at 620 nm also can be adopted which would deduct the interference of the cell debris in the samples. The data is analyzed by plotting cell number versus absorbance allowing the quantification of changes in cell proliferation. The rate of tetrazolium reduction is proportional to the rate of cell proliferation.

### Calculation:

Cytotoxicity of the sample is expressed as IC₅₀ value, the concentration which inhibits 50% of the cell growth.

$$\% Cytotoxicity = \frac{E - T}{E} \times 100$$

Where, E=Cell viability in the absence of the sample
T=Cell viability in the presence of the sample.
Hepatoprotective activity of Garcinol in HEP G2 cells
IC₅₀=13.33 ug/ml
95% CL=11.84 to 15.02 ug/ml
Hepatoprotective activity of Silymarin in HEP G2 cells
IC₅₀=36.51 ug/ml
95% CL=32.18 to 41.42 ug/ml
Lesser the IC₅₀ value, better the efficacy.

TABLE 6
[Cytotoxicity of Silymarin in Hep G2 cell line]

| Concentration (μg/ml) | % Cytotoxicity |
|---|---|
| 100.00 | 81.77 |
| 50.00 | 64.33 |
| 25.00 | 8.62 |
| 12.50 | −7.46 |
| 6.25 | −4.25 |
| 3.13 | −11.27 |
| 1.56 | −8.63 |
| 0.78 | −8.31 |
| 0.39 | 2.34 |

Sigmoidal Dose Response (variable slope)

| | |
|---|---|
| BOTTOM | 0 |
| TOP | 100 |
| IC₅₀ | 36.51 μg/ml |
| 95% CL | 32.18 to 41.42 μg/ml |
| R² | 0.9545 |

TABLE 7
[Cytotoxicity of Garcinol in Hep G2 cell line]

| Concentration (μg/ml) | % Cytotoxicity |
|---|---|
| 200.00 | 79.50 |
| 100.00 | 83.44 |
| 50.00 | 83.15 |
| 25.00 | 60.90 |
| 12.50 | 35.81 |
| 6.25 | −1.94 |
| 3.13 | −14.65 |
| 1.56 | −12.09 |
| 0.78 | −3.81 |

Sigmoidal Dose Response (variable slope)

| | |
|---|---|
| BOTTOM | 0 |
| TOP | 100 |
| IC₅₀ | 13.33 μg/ml |
| 95% CL | 11.84 to 15.02 μg/ml |
| R² | 0.9756 |

Results:

Garcinol shows comparative hepatoprotective effect like Silymarin at lower concentrations (values below 6.25 μg/ml).

While the invention has been described with reference to a preferred embodiment, it is to be clearly understood by those skilled in the art that the invention is not limited thereto. Rather, the scope of the invention is to be interpreted only in conjunction with the appended claims.

1. A method of mammalian hepatocyte protection, said method comprising step of bringing into contact mammalian hepatocytes with an effective concentration of garcinol.

2. The method according to claim 1, wherein the effective concentration of garcinol is from about 0.78 μg/ml to about 6.25 μg/ml.

3. A method of reducing increased levels of cytokine expression in mammalian models of liver damage (hepatotoxicity), said method comprising step of administering an effective amount of garcinol to said models.

4. The method according to claim 3, where in the cytokine is Transforming Growth Factor G-β1 (TGF-β1).

5. The method according to claim 3, wherein the cytokine is Tumor Necrosis Factor-α.

6. The method according to claim 3, wherein the cytokine is Interleukin-2 (IL-2).

7. The method according to claim 3, wherein the cytokine is Interleukin-4 (IL-4).

8. The method according to claim 3, wherein the cytokine is Interleukin-12 (IL-12).

9. The method according to claim 3, wherein hepatotoxicity is caused by toxin.

10. The method according to claim 3, wherein hepatotoxicity is caused by drugs.

11. The method according to claim 3, wherein hepatotoxicity is caused by ethyl alcohol.

12. A method of reducing increased levels of adhesion molecule expression in mammalian models of liver damage (hepatotoxicity), said method comprising step of administering an effective amount of garcinol to said models.

13. The method according to claim 12, wherein the adhesion molecule is intracellular Adhesion Molecule-1 (ICAM-1 or CD 52).

14. The method according to claim 12, wherein hepatotoxicity is caused by toxin.

15. The method according to claim 12, wherein hepatotoxicity is caused by drugs.

16. A method of reducing elevated levels of liver enzymes and/or bile pigments in mammalian models of liver damage (hepatotoxicity), said method comprising step of administering an effective amount of garcinol to said models.

17. The method according to claim 16, wherein hepatotoxicity is caused by toxin.

18. The method according to claim 16, wherein hepatotoxicity is caused by drugs.

19. The method according to claim 16, wherein hepatotoxicity is caused by ethyl alcohol.

20. The method according to claim 16, wherein the liver enzyme is selected from a group comprising Alanine Transaminase, Aspartate aminotransferase and Alkaline Phosphatase.

21. The method according to claim 16, wherein the bile pigment is bilirubin.

22. A method of providing hepatoprotection, said method comprising step of administering a therapeutically effective amount of garcinol to a subject in need thereof.
